(19) United States
(12) Patent Application Publication
(10) Pub. No.: US 2022/0008709 A1
(43) Pub. Date: Jan. 13, 2022

(54) INTEGRATED CONNECTOR AND DISINFECTING CAP WITH RESERVOIR

(71) Applicant: Becton, Dickinson and Company, Franklin Lakes, NJ (US)

(72) Inventor: Kevin Ryan, Whitehouse Station, NJ (US)

(73) Assignee: Becton, Dickinson and Company, Franklin Lakes, NJ (US)

(21) Appl. No.: 17/297,604

(22) PCT Filed: Nov. 26, 2019
(86) PCT No.: PCT/US2019/063233

§ 371 Date: May 27, 2021

Related U.S. Application Data
(60) Provisional application No. 62/773,551, filed on Nov. 30, 2018.

(51) Int. Cl. A61M 39/16; A61M 39/20; A61L 2/00

(52) U.S. Cl. CPC A61M 39/162; A61M 39/165; A61M 39/20; A61L 2202/123; A61L 2202/21; A61L 2202/24; A61L 2/0088

(57) ABSTRACT

Connector and disinfecting cap system includes design features that can maintain disinfectant solution for multiple connector accesses and limit the exposure time of the disinfectant solution when the connector and cap system is in an open state, for example when connector is being accessed, or a closed state, for example, when connector is not being accessed. Connector and cap system can include a cap housing, which can contain an internal disinfectant reservoir, and a connector, such as an IV needleless connector, where the cap housing is attached to the connector such that the cap can pivot in one or more axes in reference to the connector.

### CROSS-REFERENCE TO RELATED APPLICATIONS

This application claim priority under 35 USC § 119(e) from U.S. Provisional Patent Application No. 62/773,551 filed on Nov. 30, 2018, the contents of which (including all attachments filed therewith) are hereby incorporated by reference in their entirety.

### TECHNICAL FIELD

Generally, exemplary embodiments of the present disclosure relate to the fields of threaded fittings, including medical caps and medical disinfection caps, and in particular caps and/or disinfection caps for uses with IV needleless connectors.

### BACKGROUND

In the example of medical applications, various conventional caps for closing off a needleless connector while not in use have been known for some time. In order to decrease Catheter-related bloodstream infection (CRBSI) cases disinfection caps were originally disclosed in U.S. Patent Publication No. 2007/011233 which issued as U.S. Pat. No. 8,740,864 (the entire disclosures of both of which are incorporated herein by reference), and introduced on the market. Disinfection caps such as those disclosed in the U.S. Pat. No. 8,740,864 are illustrated in FIGS. 1A and 1B herein, where cap 1 includes a disinfecting pad 2 and a lid 3, and cap 4 includes a disinfecting pad 5 and lid 7, as well as threads 6 on its inner circumference 8 to interlock with needleless connector hub. On the other hand, other conventional caps may have similar features but exclude the disinfecting pad. Further improved designs for disinfection caps are disclosed in related U.S. patent application Ser. No. 15/408,278 and Ser. No. 15/408,187, both filed on Jan. 17, 2017 (the entire disclosures of both of which are incorporated herein by reference). Yet further modifications to cap designs adding further safety considerations are disclosed in related U.S. Patent Applications Nos. 62/488,266, filed on Apr. 21, 2017, 62/523,506, filed Jun. 22, 2017, and 62/623,858 filed Jan. 30, 2018 (the entire disclosures of which are incorporated herein by reference).

Currently, there are male disinfecting cap devices for disinfecting ISO594-2 type of female threaded fluid luer connectors and there are female disinfecting cap devices for disinfecting ISO594-2 type of male threaded fluid luer connectors. Such caps are designed for a one time use application for disinfection of needleless connectors. One reason that the disinfectant caps are single use, is that the caps vent (evaporate or leak) disinfection solution (such as 70% IPA) after cap is removed from the IV connector. For example, this can occur when the cap internal disinfectant solution gets exposed to air and potential microbes in the non-sterile environment (such as a hospital room), after the cap is used and removed from an IV needleless connector. After the disinfection solution diminishes due to evaporation and/or leakage, the cap can become significantly less effective or non-effective at disinfecting an IV needleless connector activation surface.

Accordingly, there is not a cap, or disinfecting cap, device designed to be reused, or for multiple uses, while the single use limitation of current caps is not ideal, for example because a clinician must have many caps on hand for disinfecting and protecting an IV connector each time after the IV connector is accessed for maintenance (for example, a Saline flush) after push medication is administered.

### SUMMARY

The matters exemplified in this description are provided to assist in a comprehensive understanding of exemplary embodiments of the disclosure. Accordingly, those of ordinary skill in the art will recognize that various changes and modifications of the embodiments described herein can be made without departing from the scope and spirit of the disclosure. Also, descriptions of well-known functions and constructions are omitted for clarity and conciseness.

As would be readily appreciated by skilled artisans in the relevant art, while descriptive terms such as “lock”, “hole”, “tip”, “hub”, “thread”, “sponge”, “protrusion”, “tab”, “slope”, “wall”, “top”, “side”, “bottom,” “upper,” “lower,” “connector,” “housing,” “disinfectant,” “disinfecting,” “rim,” “arm,” “contoured,” “barrier” and others are used throughout this specification to facilitate understanding, it is not intended to limit any components that can be used in combinations or individually to implement various aspects of the embodiments of the present disclosure.

Exemplary embodiments of the present disclosure provide an integrated connector and cap system with a disinfection solution (such as 70% IPA) reservoir design that can disinfect over multiple connector accesses.

In an exemplary implementation of the embodiments of present disclosure, an IV connector and disinfecting cap system can address the single use issue by having design features that can maintain disinfectant solution for multiple IV needleless connector accesses and limit the exposure time of the disinfectant solution when the connector and cap system is in an open state (for example, when the IV connector is being accessed) or a closed state (for example, when the IV connector is not being accessed).

According to exemplary implementations of the embodiments of the present disclosure, configuration of structural elements making up a connector and cap system comprise a housing, or a cap housing, that contains an internal disinfectant reservoir, and a connector, such as an IV needleless connector, where the cap housing is attached to the connector such that the cap can pivot in one or more axes in reference to the connector.

According to exemplary implementations of the embodiments of the present disclosure, cap housing can be attached to the connector by for example one or more arms extending from the housing and pivotally connected to the connector.

According to further exemplary implementations of the embodiments of the present disclosure, the connector comprises an actuation surface and the housing comprises a contoured opening portion such that the actuation surface can interface with the contoured opening portion of the housing when the connector is in a closed (for example, when an IV connection is not accessed) state.

According to yet further exemplary implementations of the embodiments of the present disclosure, the connector comprises a contoured barrier feature, or a protrusion that is intended to interface with the contoured opening portion of the cap housing when the connector is in an open (for example, when an IV connection is accessed) state.

According to still further exemplary implementations of the embodiments of the present disclosure, the cap housing may contain a sponge, for example soaked with a disinfecting solution, disposed within the reservoir of the cap.

According to still further exemplary implementations of the embodiments of the present disclosure, the cap housing may have cover, such as a peel lid or a seal strip, configured with respect to the contoured opening portion, for example, to maintain sterility at least of a surface of the contoured portion interfacing with the actuation surface of the connector and/or preserve the internal disinfectant contained in the reservoir, or the sponge, of the cap over shelf life prior to use.

According to still further exemplary implementations of the embodiments of the present disclosure, the cap housing may be clear, or transparent, such that an indication of disinfecting solution (for example, IPA) fill level within the cap reservoir may be observed or viewed.

According to still further exemplary implementations of the embodiments of the present disclosure, materials of the connector, or IV connector, and/or the cap housing may be selected for specific stability for use with the disinfectant solution (or solvent) to be provided in the reservoir of the cap housing.

Any combination of such exemplary implementations can be provided in the connector and cap system of the present disclosure.

### BRIEF DESCRIPTION OF THE DRAWINGS

Referring now to the drawings, wherein like reference numerals designate identical or corresponding parts throughout the several views, embodiments of the present disclosure are described as follows.

FIGS. 1A and 1B are cross sectional views of conventional caps for needleless connectors.

FIG. 2A illustrates an isometric or perspective view of a cap and connector structure in a closed, such as when for example an IV connection is not being accessed, according to exemplary embodiments of the disclosure.

FIGS. 2B and 2C illustrate cross sectional views of a cap and connector structure of FIG. 1 according to exemplary embodiments of the disclosure.

FIG. 3 diagrammatically shows an isometric or perspective view of a cap and connector structure in an open, such as when for example an IV connection can be accessed, state according to exemplary embodiments of the disclosure.

FIG. 4 illustrates a cross sectional view of a cap and connector structure of FIG. 3 according to exemplary embodiments of the disclosure.

FIG. 5 diagrammatically shows an isometric or perspective view of a cap and connector structure in a transitional (from open to close, or close to open) state according to exemplary embodiments of the disclosure.

FIG. 6 illustrates a cross sectional view of a cap and connector structure of FIG. 5 according to exemplary embodiments of the disclosure.

FIG. 7 diagrammatically shows an isometric or perspective view of a cap and connector structure in a sealed state according to exemplary embodiments of the disclosure.

FIG. 8 illustrates a cross sectional view of a cap and connector structure of FIG. 7 according to exemplary embodiments of the disclosure.

FIGS. 9A and 9B illustrate a cross sectional view of a cap and connector structure including a disinfecting component, such as a sponge of any shape, according to exemplary embodiments of the disclosure.

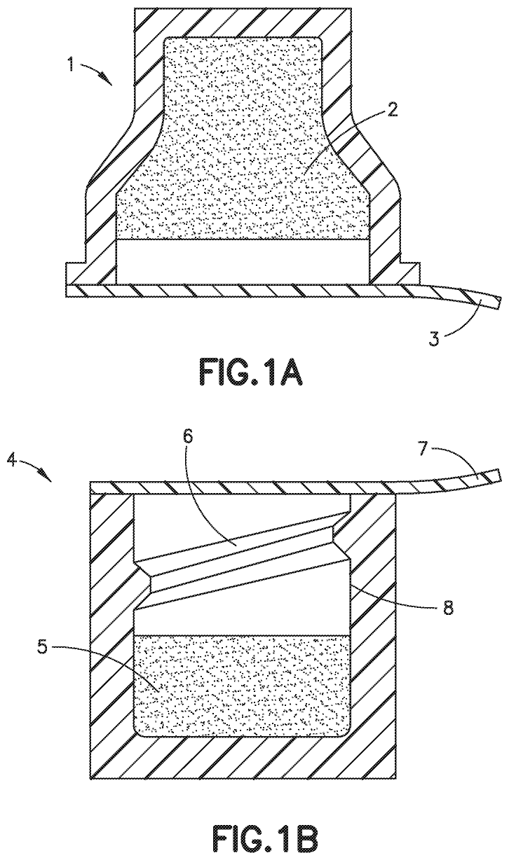

### DETAILED DESCRIPTION OF THE EXEMPLARY EMBODIMENTS

This matters exemplified in this description are provided to assist with a comprehensive understanding of exemplary embodiments with reference to the accompanying drawing figures. Accordingly, those of ordinary skill in the art will recognize that various changes and modifications of the exemplary embodiments described herein can be made within the scope of appended claims without departing from their full scope and equivalents. Also, descriptions of well-known functions and constructions are omitted for clarity and conciseness. Likewise, certain naming conventions, labels and terms as used in the context of the present disclosure are non-limiting and provided only for illustrative purposes to facilitate understanding of exemplary implementations of the exemplary embodiments.

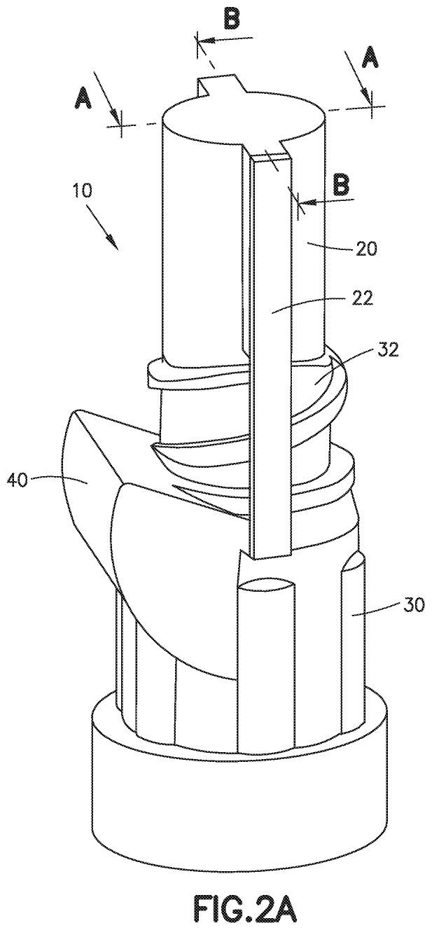

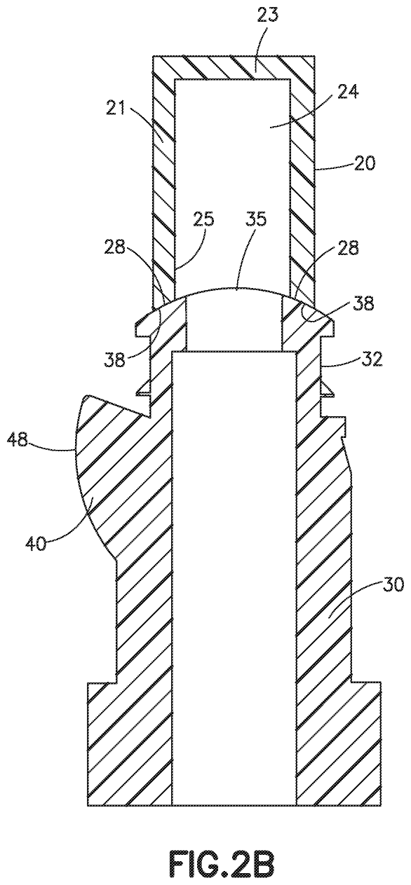

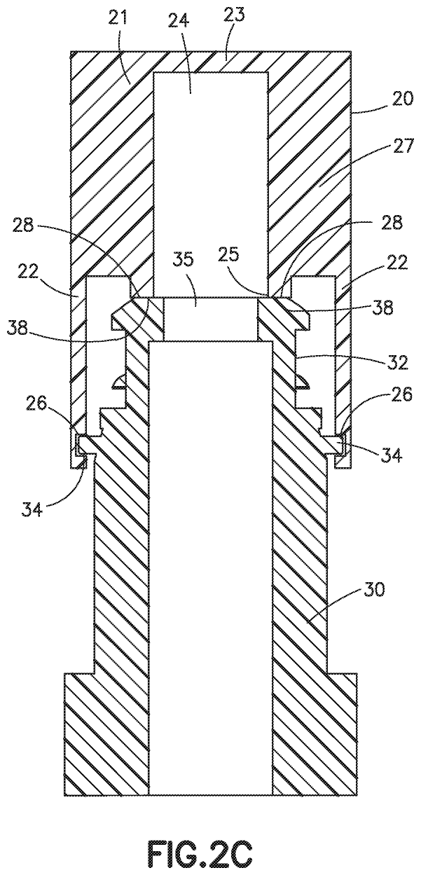

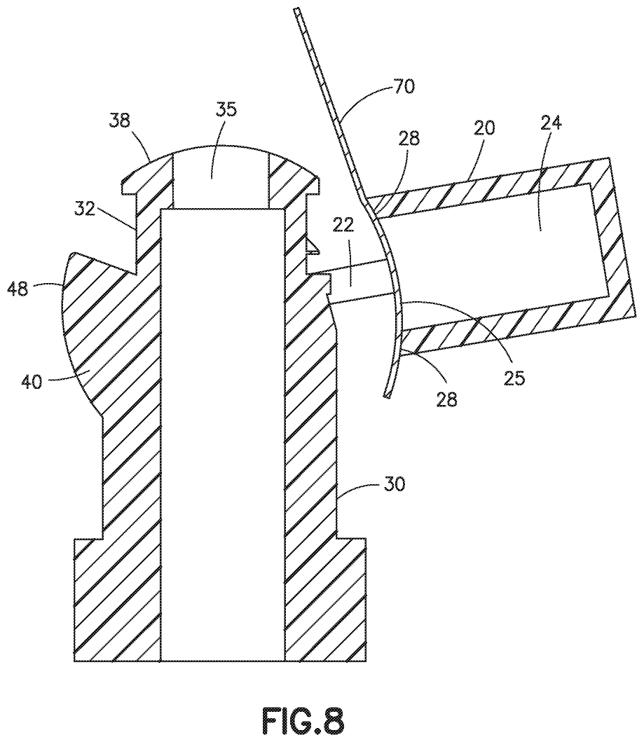

Referring to FIGS. 2A through 9B, in an exemplary implementation, design features of an integrated connector and cap device 10 comprises a cap housing 20, including a top wall 23 forming a closed ends of the housing 20 and a continuous side wall 21, such as an essentially cylindrical sidewall (although any cross sectional shape is within the scope of the disclosure) forming a cavity (or an internal disinfectant reservoir) 24 with an opening 25 into cavity 24 defined by a rim of sidewall 21 with surface 28 at an open end (for example opposite to the closed end) of cap housing 20. Cap device 10 further comprises a connector, such as an IV connector, 30 including a hub 32 with an opening 35 into connector 30 defined by a rim of hub 32 with surface, or actuation surface, 38 of tip of hub 32. Cap device 10 further comprises an attachment mechanism, such as one or more arms, 22 extending between housing 20 and connector 30 for attaching housing 20 and connector 30 such that housing 20 can pivot in one or more axes in reference to the connector 30 allowing surface 28 of cap housing 20 to selectively interface with surface 38 of connector 30.

In an exemplary implementation, arms 22 can be attached to (for example by shoulders 27), or integrally formed with, sidewall 21 of cap 20. Arms 20 can be of suitable length to extend from side wall 21 beyond open end of cap 20 to pivotally attach cap 20 to exterior surface of connector 30, for example beyond hub 32, as illustrated in FIGS. 2A and 2C. In yet further exemplary configuration, the pivotal connection can comprise a pin 34, which can be either on a surface of connector 30 or arm 22, and corresponding hole (slot, groove, or divot) 26, which can be either on a surface of connector 30 or arm 22, as further illustrated in FIGS. 2A and 2C. In still further exemplary implementation, a snap fit assembly of cap 20 with arms 22 and connector 30 can be provided, for example by the pin/slot configuration.

As illustrated in the examples of FIGS. 2A through 2C, in an exemplary implementation, surface 28 forms a contoured open end of cap housing 20 and surface 38 forms a complimentary contoured tip of hub 32, such that a contoured actuation surface 38 of connector 30 can interface with the contoured open end surface 28 of cap housing 20 when the connector (for example, IV connector) 30 is in the closed (for example IV not being accessed) state.

In yet another exemplary implementation, connector 30 of device 10 comprises a barrier (or barrier feature) 40, for example implemented as a protrusion on exterior surface of connector 30 for example below hub portion 32 and for example essentially axially perpendicular to hub 32. Barrier 40 comprises an external surface 48 configured such that housing 20 pivoting in one or more axes in reference to the connector 30 allows surface 28 of cap housing 20 to selectively interface with surface 48 of barrier 40.

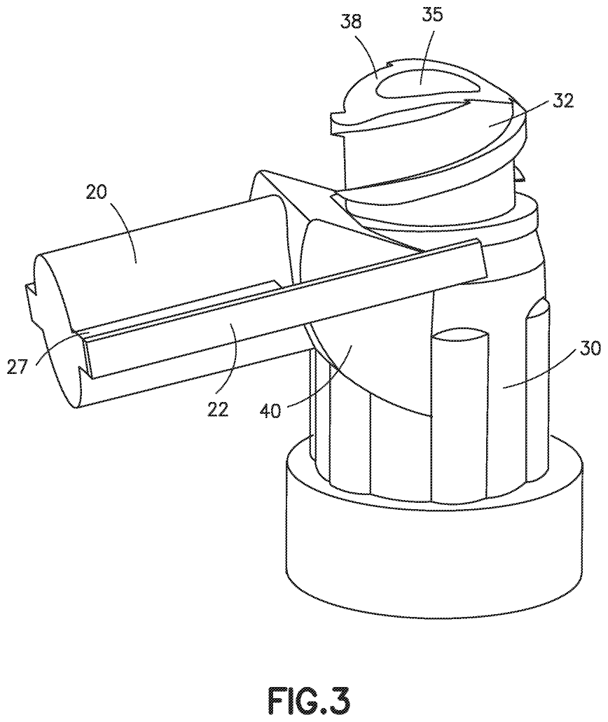

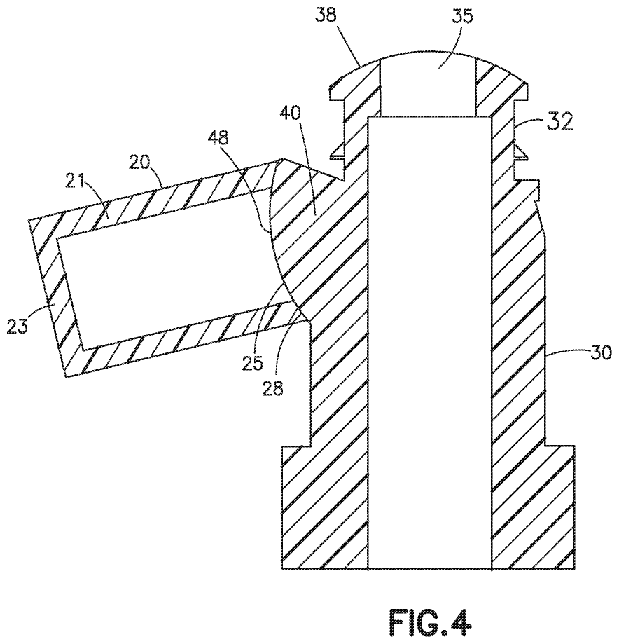

As illustrated in the examples of FIGS. 3 and 4, in an exemplary implementation, surface 48 forms a contoured portion of barrier 40, such that the contoured surface 48 of barrier 40 can interface with the contoured open end surface 28 of cap housing 20 when the connector (for example, IV connector) 30 is in the open (for example IV is being accessed) state.

Referring to FIGS. 7 through 9B, in an exemplary implementation, a peel sealing film 70 can be provided to seal the opening 25 of cap housing 20 prior to use of, for example, by attachment to surface 28 of a rim of sidewall 21 of cap housing 20 thereby maintaining sterility of reservoir 24 and/or surface 28 prior to use and/or preventing a cleaning solution that may be disposed within reservoir 24 from leaking out and/or evaporating.

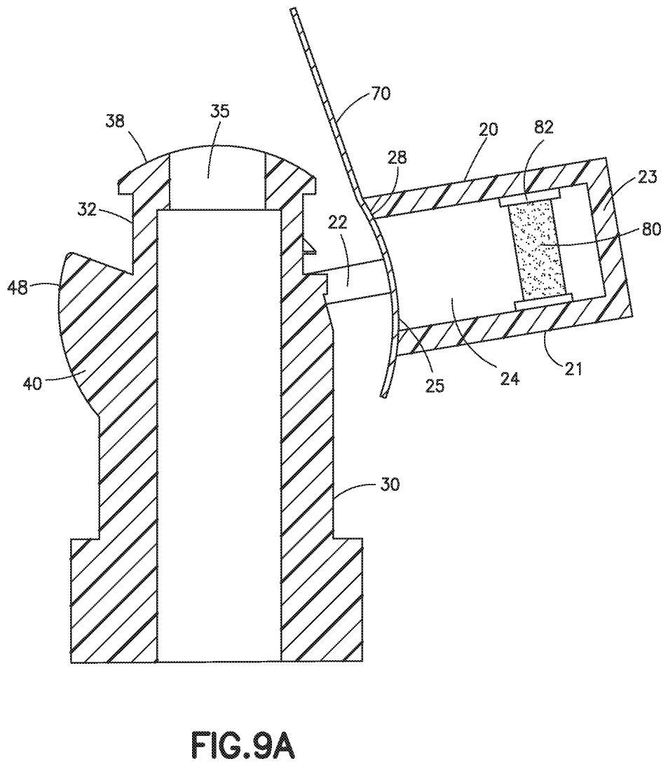

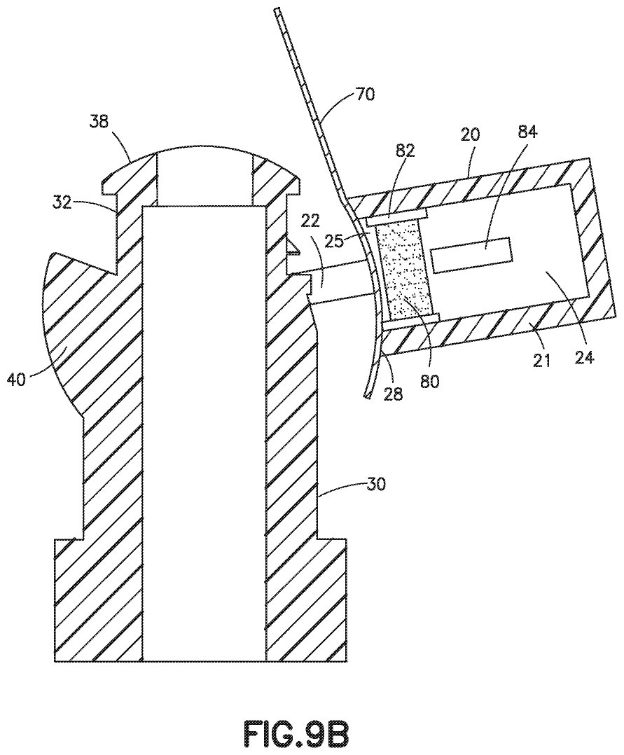

In yet another exemplary implementation, a disinfecting member or members, such as an IPA soaked sponge 80, can be provided within reservoir, or cavity, 24, for example in the proximity of inner surface of end wall 23, as illustrated in FIG. 9A, and/or within reservoir, or cavity, 24, for example in the proximity of opening 25 as illustrated in FIG. 9B, for example as described in the above-referenced prior applications. In an exemplary implementation, sponge 80 can be retained within reservoir, or cavity, 24 by one or more protrusions 82 and/or one or more protrusions 84 on interior surface of side wall 21, and/or one or more protrusions (not shown) in interior surface of end wall 23. Such one or more protrusions can retain sponge 80 within reservoir, or cavity, 24 by abutment and/or interference fit with sponge 80. In a further exemplary implementation, a peel sealing film 70 can be provided to seal the opening 25 of cap housing 20 prior to use of, for example, by attachment to surface 28 of a rim of side wall 21 of cap housing 20, thereby preventing evaporation of cleaning solution from sponge 80.

According to exemplary embodiments of the present disclosure, integrated connector and cap device 10 can implemented with design functions covering at least four states including a connector (such as an IV Connector) closed state, a connector (such as an IV Connector) open state, a transitional state, and a sealed state.

Referring to exemplary illustrations of FIGS. 2A-2C, when integrated connector and cap device 10 is in the closed state (for example an IV un accessed state), cap housing 20 is in contact with and covering the connector 30 access surface 38. For example, while in this state, cap housing 20 contoured open end surface 28 and the connector 30 surface (for example IV connector contoured actuation surface) 38 are designed such that an open area between the two are minimized or optimized (for example, by appropriate configuration of arm(s) 22, pin 34 and divot 26) such that the disinfection solution leakage or evaporation out of reservoir 24 is reduced and disinfection solution is maintained. For example, this can facilitate the cap 20 to remain effective for a prolonged time period while in this closed state. In addition while in this closed state, the cap 30 disinfection solution contained in reservoir 24 can be in contact with the connector 30 and can be actively disinfecting surface 38 of connector 30.

Referring to exemplary illustrations of FIGS. 3 and 4, when integrated connector and cap device 10 is in the open state (for example an IV accessed state), the cap housing 20 is in contact with the connector 30 barrier feature 40 surface 48. For example, while in this state, the cap housing 20 contoured open end surface 28 and the connector 30 barrier feature 40 contoured surface 48 are designed such that the open area between the two are minimized or optimized (for example, by appropriate configuration of arm(s) 22, pin 34 and divot 26) such that the disinfection solution leakage or evaporation out of reservoir 24 is reduced and disinfection solution is maintained. For example, this can facilitate the cap 20 to remain effective for a prolonged time period while in this open state. In addition, while in this is open state, the connector 30, or hub 32 of connector 30, can be accessed with for example an IV line or syringe pursuant to a normal or conventional clinical practice.

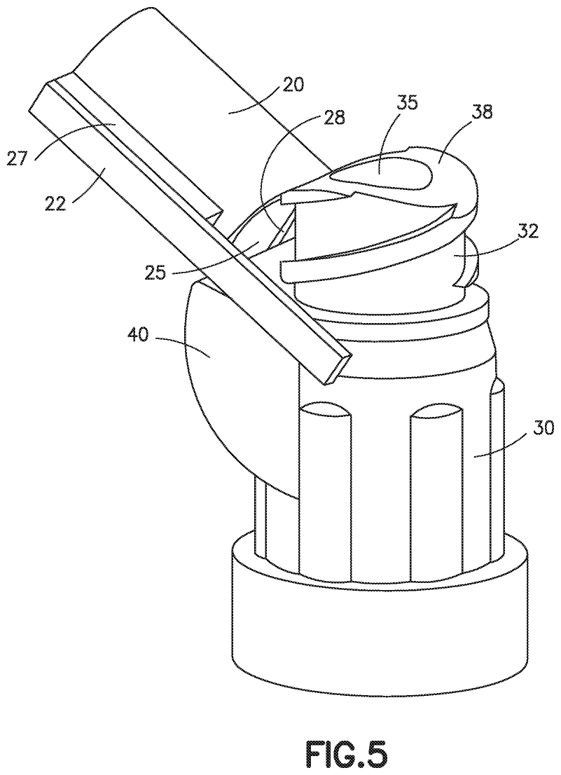

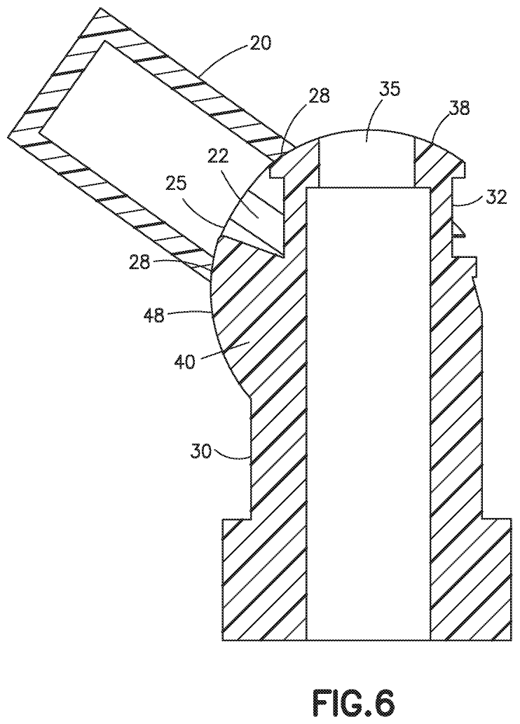

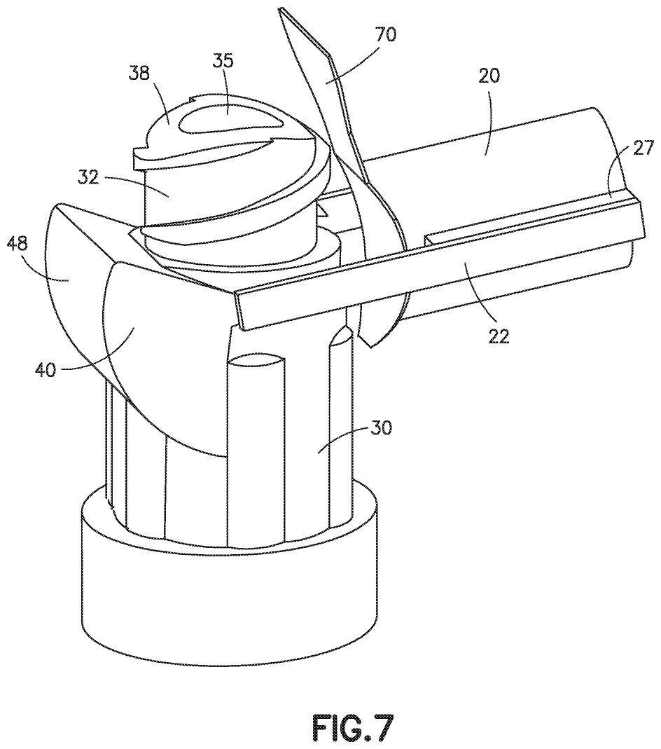

Referring to exemplary illustrations of FIGS. 5 and 6, integrated connector and cap device 10 undergoes the transitional state only momentarily for example when clinician toggles the cap housing 20 to the desired open (see for example FIG. 2A) or closed (see for example FIG. 3) state by rotating or sliding the cap housing 20 to either position. Since the cap housing is only momentarily in this transition state, reservoir 24 is only momentarily exposed via opening 25, and therefore, the evaporation loss of the disinfection solution out of reservoir 24 can be minimized.

Referring to exemplary illustrations of FIGS. 7 through 9B, when integrated connector and cap device 10 is in the sealed state, for example before product use, the disinfection solution is maintained over shelf life within reservoir 25 by being sealed therein, for example by a seal 70 which can be removed by clinician prior to use.

According to yet another exemplary implementation of the embodiments of the disclosure, the cap 20 does not push disinfectant solution into the connector 30 during capping application, at least because the connector 30 is not squeezing into (causing increased disinfectant solution pressure) inside (within cavity 24) of the cap housing 20, as illustrated in the example of FIGS. 2A through 2C.

While the present disclosure has been shown and described with reference to certain exemplary embodiments thereof, it will be understood by those skilled in the art that various changes in form and details may be made therein without departing from the spirit and scope of the embodiments of the present disclosure. For example, a disinfection sponge can comprise any suitable disinfecting or other application-specific substance, and can be made of any suitable material. Also, the cap housing and/or the connector can be single shot molded, or made by other suitable process. Furthermore, any of the features or elements of any exemplary implementations of the embodiments of the present disclosure as describes above and illustrated in the drawing figures can be implemented individually or in any combination(s) as would be readily appreciated by skilled artisans without departing from the spirit and scope of the embodiments of the present disclosure.

In addition, the included drawing figures further describe non-limiting examples of implementations of certain exemplary embodiments of the present disclosure and aid in the description of technology associated therewith. Any specific or relative dimensions or measurements provided in the drawings other as noted above are exemplary and not intended to limit the scope or content of the inventive design or methodology as understood by artisans skilled in the relevant field of disclosure.

Other objects, advantages and salient features of the disclosure will become apparent to those skilled in the art from the details provided, which, taken in conjunction with the annexed drawing figures, disclose exemplary embodiments of the disclosure.

1. A system comprising:
a cap housing comprising a reservoir for storing a disinfecting solution and an open end with an opening into said reservoir, said open end comprising a contoured opening portion;
a connector comprising a contoured actuation surface on a tip of said connector and a barrier on a side of said connector, said barrier comprising a contoured external surface; and
an attachment mechanism pivotally connecting said cap housing to said connector such that said cap housing selectively interfaces with said connector and said barrier,
wherein:
when said cap housing closes said connector, said contoured opening portion of said cap housing interfaces with said contoured actuation surface of said connector, and
when said cap housing opens said connector, said contoured opening portion of said cap housing interfaces with said contoured external surface of said barrier.

2. The system of claim 1 wherein said housing comprises:
a continuous sidewall,
a top wall, and
a closed end formed by said top wall,
wherein:
said reservoir comprises a cavity within said housing formed by interior surfaces of said top wall and said sidewall, said open end is formed by a rim of said sidewall, and a surface of said rim forms said contoured opening portion of said cap housing.

3. The system of claim 1, wherein said connector is an IV needleless connector.

4. The system of claim 1, wherein said cap housing pivots in one or more axes in reference to said connector.

5. The system of claim 1, wherein said attachment mechanism comprises one or more arms extending from said cap housing and fixed with respect to said housing, and pivotally connected to an exterior surface of said connector.

6. The system of claim 1, further comprising at least one disinfection sponge configured within said reservoir.

7. The system of claim 1, further comprising a removable cover configured on said contoured opening portion sealing said opening into said reservoir.

8. The system of claim 1, wherein said cap housing comprises a clear portion enabling view of a fill level of said disinfecting solution within said reservoir.

9. The system of claim 1, wherein said connector comprises a hub having said tip, and said contoured actuation surface forming said tip of said hub.

10. The system of claim 9, wherein said barrier comprising said contoured external surface is essentially axially perpendicular to said hub comprising said contoured actuation surface.

11. The system of claim 2, wherein a removable cover is configured on said contoured opening portion by attachment to a surface of said rim of said sidewall of said cap housing.

12. The system of claim 1, wherein said contoured opening portion of said cap housing interfaces with said contoured actuation surface of said connector such that a first open area between said contoured opening portion and said contoured actuation surface is minimized.

13. The system of claim 1, wherein said contoured opening portion of said cap housing interfaces with said contoured external surface of said barrier such that a second open area between said contoured opening portion and said contoured external surface is minimized.

14. The system of claim 12, wherein at least one of said first and second open areas are minimized by an optimized configuration of said attaching mechanism with respect to said cap housing and said connector.

15. The system of claim 14, wherein said optimized configuration of said attaching mechanism comprises optimized configuration of said pivot connection of said cap housing to said connector.

16. The system of claim 2, wherein said connector is an IV needleless connector.

17. The system of claim 2, wherein said cap housing pivots in one or more axes in reference to said connector.

18. The system of claim 2, wherein said attachment mechanism comprises one or more arms extending from said cap housing and fixed with respect to said housing, and pivotally connected to an exterior surface of said connector.

19. The system of claim 2, further comprising at least one disinfection sponge configured within said reservoir.

20. The system of claim 2, further comprising a removable cover configured on said contoured opening portion sealing said opening into said reservoir.
